(19) United States
(12) Patent Application Publication
(10) Pub. No.: US 2020/0191738 A1
(43) Pub. Date: Jun. 18, 2020

(54) USE OF C4BP-ALPHA PROTEIN IN HUMAN SERUM TO PREDICT CLOPIDOGREL RESISTANCE AND ITS CLINICAL APPLICATION AS A NOVEL BIOMARKER

(71) Applicant: NANJING FIRST HOSPITAL, Nanjing (CN)

(72) Inventors: Hongguang XIE, NANJING (CN); Jinzi JI, NANJING (CN); Beibei HUANG, NANJING (CN); Ting TAI, NANJING (CN); Qiongyu MI, NANJING (CN); Wenjian XIE, NANJING (CN)

(73) Assignee: NANJING FIRST HOSPITAL

(21) Appl. No.: 16/709,822

(22) Filed: Dec. 10, 2019

(30) Foreign Application Priority Data
Dec. 12, 2018 (CN) 201811515436.9

(51) Int. Cl. G01N 27/04; G01N 33/49; G01N 33/50; C12Q 1/56

(52) U.S. Cl. CPC G01N 27/04; G01N 33/49; C12Q 1/6827; C12Q 1/56; G01N 2800/52; G01N 33/5008

(57) ABSTRACT

The present invention discloses a novel biomarker for predicting clopidogrel resistance and a clinical application thereof. Application of C4BPα protein in human serum as an assay target or standard substance in the preparation of an assay kit for predicting clopidogrel resistance is disclosed. Clinically, the detection of C4BPα in human serum can predict clopidogrel resistance, thereby providing a novel assay for guiding rational use of clopidogrel or drug switching, and helping clinicians to diagnose or predict the clinical efficacy of clopidogrel.

### CROSS-REFERENCE TO RELATED APPLICATIONS

This application claims priority to Chinese Patent Application No. 201811515436.9 with a filing date of Dec. 12, 2018. The content of the aforementioned applications, including any intervening amendments thereto, are incorporated herein by reference.

### TECHNICAL FIELD

The present invention belongs to the field of biological assay and relates to use of the human serum protein C4BPα (a naturally occurring complement inhibitor present in human plasma) as a novel biomarker to predict clopidogrel resistance and a clinical application thereof.

### BACKGROUND OF THE PRESENT INVENTION

As an antiplatelet drug, clopidogrel, in combination with aspirin, is frequently prescribed for the secondary prevention of recurrent myocardial ischemia or stent thrombosis for patients with acute coronary syndrome or those undergoing percutaneous coronary intervention (PCI) for stent implantation. The dual antiplatelet therapy strategy (combined use of clopidogrel and aspirin) is the gold standard of the antiplatelet therapy after stent implantation for the PCI patients, and is recognized or accepted globally.

Clopidogrel resistance is defined as poor or no response to clopidogrel in some patients taking the drug at the recommended dose due to various causes, known or unknown, with a variable prevalence ranged from 10% to 45% of the patients taking the drug.

In clinical practice, the extent of inhibition of ADP-induced platelet aggregation before and after dosing is frequently used to judge clopidogrel resistance and to guide dosage adjustments or drug switching. However, such an assay has faced a challenge in its reproducibility and accuracy.

In addition, use of the CYP2C19 genotypes to guide dose adjustment of clopidogrel or drug switching is recommended by the US FDA for patients with an indication of clopidogrel. However, the patient populations are generally categorized as the tree genotype groups (wild-type homozygote, wild-type/variant heterozygote, and variant homozygote) in a qualitative manner, and the CYP2C19 genotypes contribute less to the overall interindividual variation of clopidogrel efficacy (about 12%). For example, a recent clinical research study showed that the diagnostic sensitivity of distinguishing the presence or absence of clopidogrel resistance by using the CYP2C19 genotype is only 30.4%, with the area under the ROC curve being 0.617, indicating limited clinical diagnostic value or predictive significance (data not published). It has been demonstrated that many causes are involved in the presence of clopidogrel resistance, but that the mechanisms underlying clopidogrel resistance are largely unknown. Currently, there is no more effective biomarker for predicting clopidogrel resistance. Therefore, discovery and validation of novel biomarkers are still needed for clinical practice.

### SUMMARY OF PRESENT INVENTION

The purpose of the present invention is to provide a novel biomarker for predicting clopidogrel resistance and a clinical application thereof with respect to the above shortcomings of the related prior art.

The purpose of the present invention can be achieved by the following technical solutions:

An application of human serum complement inhibitor C4-binding protein alpha (in short, C4BP-alpha, C4BPα, C4BP-α, C4BPα, or C4BP-α) as an assay target or standard substance in preparation of an assay kit for predicting clopidogrel resistance is provided.

An application of a reagent for detecting C4BPα protein in human serum in preparation of a kit for predicting clopidogrel resistance is provided.

A kit for predicting clopidogrel resistance comprises an antibody for detecting C4BPα protein in human serum.

The kit for detecting C4BPα in human serum preferably comprises related reagents for detecting the C4BPα protein in human serum by an ELISA method.

### BENEFICIAL EFFECTS

The present invention discovers that the levels of C4BPα (protein) in human serum can accurately predict clopidogrel resistance (see FIG. 1). Based on the above discovery, the present invention provides a C4BPα protein in human serum as a novel biomarker for predicting clopidogrel resistance. The biomarker, as a detection target, can predict or assist in judging the clinical efficacy or prognosis of patients after taking the drug. The results of clinical research studies show that the area under the ROC curve of the detection of C4BPα protein in the human serum for the clopidogrel resistance is 0.852, the sensitivity is 88.6%, and the specificity is 75% (see FIG. 2).

### DESCRIPTION OF THE DRAWINGS

FIG. 1 shows protein quantification of C4BPα in human serum and the marked difference between the two groups of patients who were sensitive (S) or resistant (R) to clopidogrel therapy (mean±SEM; 19.1±1.3 vs. 11.0±0.5; S vs. R; p<0.0001); and

FIG. 2 is the ROC curve of diagnosis of clopidogrel resistance by using C4BPα protein in human serum samples.

### DETAILED DESCRIPTION OF PREFERRED EMBODIMENTS

Use of the human serum C4BPα protein assay to predict clopidogrel resistance in the clinical settings.

A commercially available ELISA kit for detecting human serum C4BPα protein levels consists of the following components (product of Cloud-Clone Corp., Wuhan, Hubei, China):

deionized water

PBS (1×)

96-well plate (pre-coated)

plate film

human C4BPα protein standard

diluent of the human C4BPα protein standard from the above step [0022]

assay solution A

diluent of assay solution A (as a working solution)

assay solution B

diluent of assay solution B (as a working solution)

TMB substrate solution

stop solution

concentrated washing solution (30×)

Assay details of the kit for the measurement of the human serum C4BPα

The key steps are outlined as below (from the protocol of the kit manufacturer):

1. Preparation of serum C4BPα standard calibration curve (prepared within 15 min before use):

(1) 1 mL of diluent for C4BPα standard is added to a vial of the human C4BPα protein standard; the mixture is placed at room temperature for 10 min; and thereafter, the mixture is vortex-mixed for thorough dissolution.

(2) Eight diluted calibration samples of the human C4BPα protein standard are prepared at 10, 5, 2.5, 1.25, 0.625, 0.312, 0.156, and 0 ng/mL, respectively, through strictly serial dilution, all of which are used to establish the C4BPα calibration curve for quantification.

2. Sample processing:

(1) The frozen human serum samples are melted on ice, shaken gently, and mixed thoroughly;

(2) 10 μL of the patient serum is added to 90 μL of PBS, and mixed thoroughly;

(3) 10 μL of the (2) solution (diluted, see above) is added to 990 μL of PBS, and mixed thoroughly;

(4) 10 μL of the (3) solution (diluted, see above) is added to 990 μL of PBS, and mixed thoroughly to make the human serum samples diluted, with a final dilution of 100,000 times (that is, 10×100×100).

3. Quantification of the C4BPα protein in human serum samples:

(1) 100 μl of the diluted C4BPα standard serial concentrations (at 8 levels, see above for details) each is added, in sequence, to the 8 designated wells for preparation of the C4BPα calibration curve, whereas 100 μl of the diluted serum samples from each patient is added to the remaining wells for quantification. And then the film-coated ELISA plate is incubated in water bath at 37° C. for 2 h.

(2) The liquid in each plate well is discarded.

(3) 100 μL of working assay solution A (prepared if needed) is added to each well (see above), and then the ELISA plate is coated with the plate film again for incubation (see above) at 37° C. for 1 h.

(4) The liquid in each plate well is discarded again after each well is washed with 350 μL of washing solution and soaked for 2 min, until the liquid in each plate well is dry.

(5) The ELISA plate well is washed 3 times using the washing solution and is discarded each time.

(6) 100 μL of working assay solution B (prepared if needed) is added to each plate well, and the film-coated plate is in water bath at 37° C. for 30 min.

(7) The liquid in each plate well is discarded and dried, and the plate is washed 5 times as detailed as above step (5) in [0046].

(8) 90 μL of TMB substrate solution is added to each plate well, and the plate is coated with the plate film to develop color at 37° C. in the dark for 20 min (the plate wells designated for the C4BPα standards turn blue).

(9) 50 μL of stop solution is added to each well to cease the reaction. Starting at this time point, the color of the final solution is changed from blue to yellow immediately.

(10) The TECAN analyzer is used to measure the optical density (OD) value of each well at 450 nm.

(11) The C4BPα concentration (ng/mL) of each diluted serum sample is calculated according to the C4BPα standard calibration curve, the OD value of each serum sample examined, and the dilution times of serum samples.

Evaluation of Clinical Utility

A total of 88 PCI patients subjected to coronary angiography reexamination after stent implantation took clopidogrel 75 mg per day for at least 6 months (concomitant use of aspirin). Approximately 1 mL of fasting venous blood of each patient was withdrawn in the morning before angiography, and citrate anticoagulation (tube with blue cover) was used to determine the extent of ADP-induced whole-blood platelet aggregation, as measured with electronic resistance, Ω, or ohm; the remaining whole blood was centrifuged to separate the supernatant; and the supernatant was frozen in a refrigerator at −80° C. until analysis.

The patients were divided into two groups according to their measured ADP-induced whole-blood platelet aggregation (see above), wherein:

The negative group: 44 patients sensitive to clopidogrel, with their platelet aggregation of 0-1 ohm;

The positive group: 44 patients resistant to clopidogrel, with their platelet aggregation of ≥10 ohm.

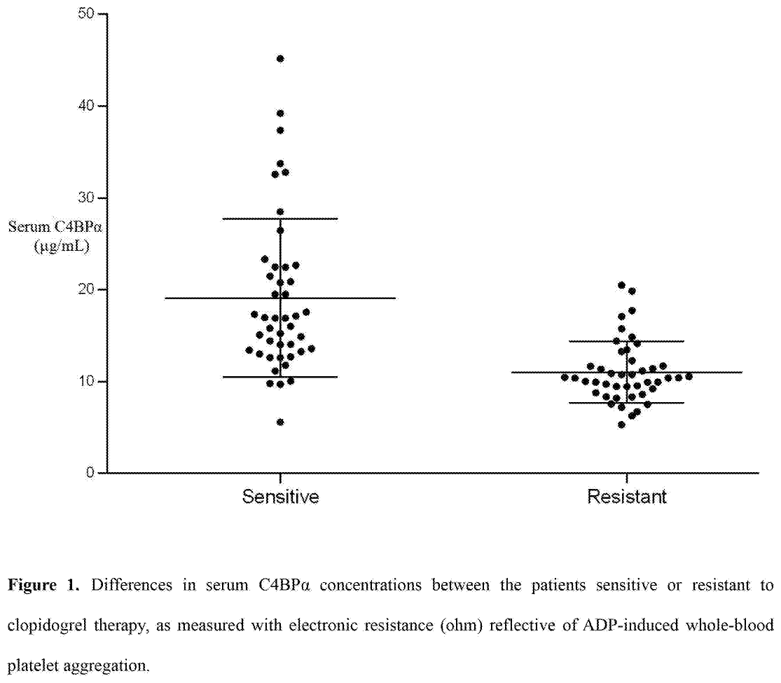

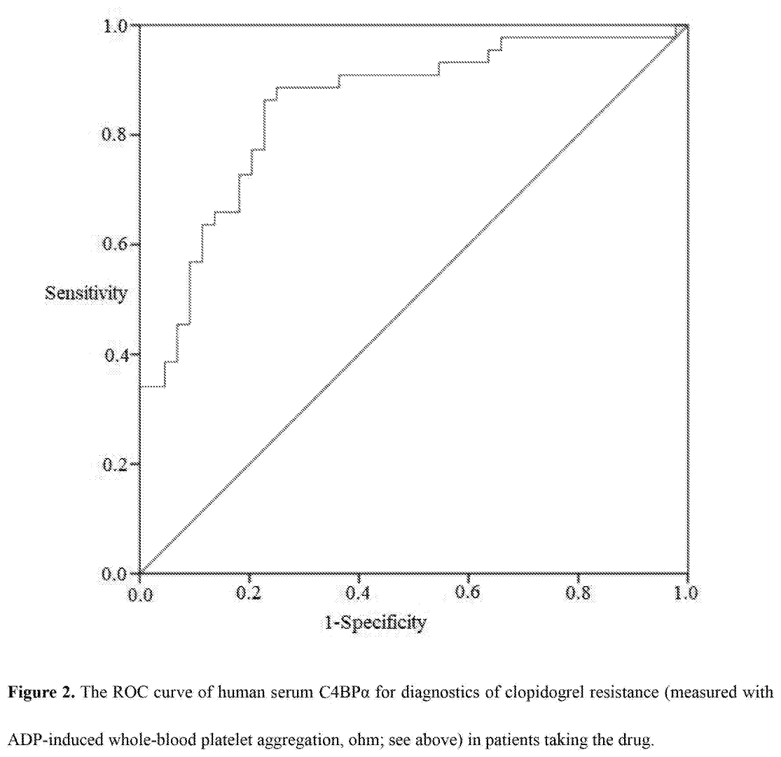

The serum of the patients was extracted according to the above experimental method, and the serum C4BPα protein concentrations were measured using the commercially available ELISA kit (see above). In the present invention, the content of the C4BPα protein in the serum samples of 44 patients with clopidogrel sensitivity was used as the group negative for clopidogrel resistance, whereas the content of the C4BPα protein in the serum samples of another 44 patients with clopidogrel resistance was used as the group positive for clopidogrel resistance; the differences between the two groups in serum C4BPα protein levels were analyzed and compared statistically (see FIG. 1). The ROC curve of diagnosing clopidogrel resistance by the content of the C4BPα protein in human serum is established (see FIG. 2). The results show that the area under the ROC curve (AUC) is 0.852 (95% CI: 0.771-0.933; p<0.0001). The sensitivity (true positive rate) to clopidogrel resistance diagnosis is 88.6%, the specificity (true negative rate) is 75%, and Youden index J is 0.636. The optimal cutoff value for the diagnosis of clopidogrel resistance is 11.733 g/mL

We claim:

1. An application of C4BPα protein in human serum as a detection target or standard substance in preparation of a kit for predicting clopidogrel resistance in clinical settings.

2. An application of related reagents for detecting C4BPα protein in the human serum in preparation of a kit for predicting clopidogrel resistance.
